(19) United States
(12) Patent Application Publication
(10) Pub. No.: US 2008/0306038 A1
(43) Pub. Date: Dec. 11, 2008

(54) Compositions and Methods for Modulating Inflammation Using Fluoroquinolones

(76) Inventors: Jinzhong Zhang, Pittsford, NY (US); Keith Wayne Ward, Ontario, NY (US)

Correspondence Address: Bausch & Lomb Incorporated, One Bausch & Lomb Place, Rochester, NY, 14604-2701, US

(21) Appl. No.: 12/124,578

(22) Filed: May 21, 2008

Related U.S. Application Data
(60) Provisional application No. 60/943,154, filed on Jun. 11, 2007.

(51) Int. Cl. A61K 31/58; A61K 31/55; A61K 31/551; A61P 27/02; A61K 31/573

(52) U.S. Cl. 514/174; 514/217.07; 514/218; 514/180

(57) ABSTRACT

Compositions for modulating an inflammation comprise a fluoroquinolone having one of Formulae I-VIII. Methods for modulating an inflammation comprise administering such compositions to a subject in need thereof. The compositions and methods are suitable for modulating an ocular or ophthalmic inflammation, including uveitis, vernal keratoconjunctivitis, or inflammation associated with contact lens-associated corneal infiltrates.

### CROSS REFERENCE

This application claims the benefit of Provisional Patent Application No. 60/943,154 filed Jun. 11, 2007 which is incorporated by reference herein.

### BACKGROUND OF THE INVENTION

The present invention relates to compositions and methods for modulating inflammation using fluoroquinolones. In particular, the present invention relates to compositions and methods for modulating ocular or ophthalmic inflammation using fluoroquinolones. In addition, the present invention relates to compositions and methods for treating, controlling, reducing, or ameliorating ocular or ophthalmic infections and their resulting inflammation using fluoroquinolones.

The interface between the body and its environment is large, and thus presents many potential opportunities for invasion by environmental virulent pathogens. The outer tissues of the eye constitute parts of this interface, and thus, the eye and its surrounding tissues are also vulnerable to virulent microorganisms, the invasion and uncontrolled growth of which cause various types of ophthalmic infections, such as blepharitis, conjunctivitis, keratitis, or trachoma, which can result in serious impairment of vision if untreated. The common types of microorganisms causing ophthalmic infections are viruses, bacteria, and fungi. These microorganisms may directly invade the surface of the eye, or permeate into the globe of the eye through trauma or surgery, or transmit into the eye through the blood stream or lymphatic system as a consequence of a systemic disease. The microorganisms may attack any part of the eye structure, including the conjunctiva, the cornea, the uvea, the vitreous body, the retina, and the optic nerve. Ocular or ophthalmic infections can cause severe pain, swollen and red tissues in or around the eye, and blurred and decreased vision.

The body's innate cascade is activated soon after invasion by a foreign pathogen begins. Leukocytes (neutrophils, eosinophils, basophils, monocytes, and macrophages) are attracted to the site of infection in an attempt to eliminate the foreign pathogen through phagocytosis. Leukocytes and some affected tissue cells are activated by the pathogens to synthesize and release proinflammatory cytokines such as IL-1β, IL-3, IL-5, IL-6, IL-8, TNF-α (tumor necrosis factor-α), GM-CSF (granulocyte-macrophage colony-stimulating factor), and MCP-1 (monocyte chemotactic protein-1). These released cytokines then further attract more immune cells to the infected site, amplifying the response of the immune system to defend the host against the foreign pathogen. For example, IL-8 and MCP-1 are potent chemoattractants for, and activators of, neutrophils and monocytes, respectively, while GM-CSF prolongs the survival of these cells and increases their response to other proinflammatory agonists. TNF-α can activate both types of cell and can stimulate further release of IL-8 and MCP-1 from them. IL-1 and TNF-α are potent chemoattractants for T and B lymphocytes, which are activated to produce antibodies against the foreign pathogen.

Although an inflammatory response is essential to clear pathogens from the site of infection, a prolonged or overactive inflammatory response can be damaging to the surrounding tissues. For example, inflammation causes the blood vessels at the infected site to dilate to increase blood flow to the site. As a result, these dilated vessels become leaky. After prolonged inflammation, the leaky vessels can produce serious edema in, and impair the proper functioning of, the surrounding tissues (see; e.g., V. W. M. van Hinsbergh, Arteriosclerosis, Thrombosis, and Vascular Biology, Vol. 17, 1018 (1997)). In addition, a continued dominating presence of macrophages at the injured site continues the production of toxins (such as reactive oxygen species) and matrix-degrading enzymes (such as matrix metalloproteinases) by these cells, which are injurious to both the pathogen and the host's tissues. Therefore, a prolonged or overactive inflammation should be controlled to limit the unintended damages to the body and to hasten the body's recovery process.

Glucocorticoids (also referred to herein as “corticosteroids”) represent one of the most effective clinical treatment for a range of inflammatory conditions, including acute inflammation. However, steroidal drugs can have side effects that threaten the overall health of the patient.

It is known that certain glucocorticoids have a greater potential for elevating intraocular pressure (“IOP”) than other compounds in this class. For example, it is known that prednisolone, which is a very potent ocular anti-inflammatory agent, has a greater tendency to elevate IOP than fluorometholone, which has moderate ocular anti-inflammatory activity. It is also known that the risk of IOP elevations associated with the topical ophthalmic use of glucocorticoids increases over time. In other words, the chronic (i.e., long-term) use of these agents increases the risk of significant IOP elevations. Unlike acute ocular inflammation associated with physical trauma or infection of the outer surface of the anterior portion of the eye, which requires short-term therapy on the order of a few weeks, infection and inflammation of the posterior portion of the eye can require treatment for extended periods of time, generally several months or more. This chronic use of corticosteroids significantly increases the risk of IOP elevations. In addition, use of corticosteroids is also known to increase the risk of cataract formation in a dose- and duration-dependent manner. Once cataracts develop, they may progress despite discontinuation of corticosteroid therapy.

Chronic administration of glucocorticoids also can lead to drug-induced osteoporosis by suppressing intestinal calcium absorption and inhibiting bone formation. Other adverse side effects of chronic administration of glucocorticoids include hypertension, hyperglycemia, hyperlipidemia (increased levels of triglycerides) and hypercholesterolemia (increased levels of cholesterol) because of the effects of these drugs on the body metabolic processes.

Therefore, there is a continued need to provide improved pharmaceutical compounds, compositions, and methods for modulating inflammation. It is also desirable to provide pharmaceutical compounds, compositions, and methods for treating, controlling, reducing, or ameliorating infections and their inflammatory sequelae. In particular, it is also very desirable to provide such compounds, compositions, and methods for modulating ocular or ophthalmic inflammation.

### SUMMARY OF THE INVENTION

In general, the present invention provides compositions and methods for modulating inflammation using fluoroquinolones.

In one aspect, the present invention provides compositions and methods for modulating ocular or ophthalmic inflammation using a novel fluoroquinolone.

In another aspect, such inflammation is anterior uveitis or vernal keratoconjunctivitis.

In another aspect, the present invention provides compositions comprising and methods for modulating ocular or ophthalmic inflammation using a fluoroquinolone having Formula 1 or a salt thereof

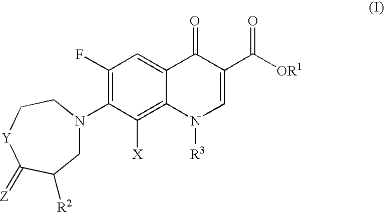

wherein R¹ is selected from the group consisting of hydrogen, unsubstituted lower alkyl groups, substituted lower alkyl groups, cycloalkyl groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, and groups that can be hydrolyzed in living bodies; R² is selected from the group consisting of hydrogen, unsubstituted amino group, and amino groups substituted with one or two lower alkyl groups; R³ is selected from the group consisting of hydrogen, unsubstituted lower alkyl groups, substituted lower alkyl groups, cycloalkyl groups, unsubstituted lower alkoxy groups, substituted lower alkoxy groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, unsubstituted C₅-C₂₄ aryloxy groups, substituted C₅-C₂₄ aryloxy groups, unsubstituted C₅-C₂₄ heteroaryloxy groups, substituted C₅-C₂₄ heteroaryloxy groups, and groups that can be hydrolyzed in living bodies; X is selected from the group consisting of halogen atoms; Y is selected from the group consisting of CH₂, O, S, SO, SO₂, and NR⁴, wherein R⁴ is selected from the group consisting of hydrogen, unsubstituted lower alkyl groups, substituted lower alkyl groups, and cycloalkyl groups; and Z is selected from the group consisting of oxygen and two hydrogen atoms.

In still another aspect, the present invention provides compositions and methods for treating, controlling, reducing, or ameliorating an ocular or ophthalmic infection and its inflammatory sequelae in a subject, using a fluoroquinolone having Formula I or a salt thereof.

In yet another aspect, such an infection is caused by bacteria, viruses, fungi, or protozoans.

In a further aspect, such an ophthalmic infection is selected from the group consisting of blepharitis, conjunctivitis, keratitis, trachoma, and combinations thereof.

In still another aspect, the present invention provides a method for modulating an inflammation in a subject. The method comprises administering into the subject an effective amount of the fluoroquinolone having Formula I or a salt thereof to modulate the inflammation.

In yet another aspect, the present invention provides a method for modulating an ocular or ophthalmic inflammation in a subject. The method comprises administering topically or intraocularly into the subject an effective amount of the fluoroquinolone having Formula I or a salt thereof to modulate the inflammation.

Other features and advantages of the present invention will become apparent from the following detailed description and claims and the appended figures.

### BRIEF DESCRIPTION OF THE FIGURES

FIG. 1 shows the effect of moxifloxacin and compound having Formula IV (“BOL-303224-A”) on LPS-simulated GM-CSF, IL-1β, and IL-8, IP-10, MCP-1, and MIP-1α production in THP-1 monocytes.

FIG. 2 shows the effect of moxifloxain and compound having Formula IV on LPS-stimulated G-CSF, IL-1α, IL-1ra, IL-6, and VEGF production in THP-1 monocytes.

FIG. 3 shows the effect of moxifloxacin and compound having Formula IV on LPS-simulated IL-12p40 production in THP-1 monocytes.

### DETAILED DESCRIPTION OF THE INVENTION

As used herein, the term “lower alkyl” or “lower alkyl group” means a C₁-C₁₅ linear- or branched-chain saturated aliphatic hydrocarbon monovalent group, which may be unsubstituted or substituted. The group may be partially or completely substituted with halogen atoms (F, Cl, Br, or I). Non-limiting examples of lower alkyl groups include methyl, ethyl, n-propyl, 1-methylethyl (isopropyl), n-butyl, n-pentyl, 1,1-dimethylethyl (t-butyl), and the like. It may be abbreviated as “Alk”. Preferably, a lower alkyl group comprises 1-10 carbon atoms. More preferably, a lower alkyl group comprises 1-5 carbon atoms.

As used herein, the term “lower alkoxy” or “lower alkoxy group” means a C₁-C₁₅ linear- or branched-chain saturated aliphatic alkoxy monovalent group, which may be unsubstituted or substituted. The group may be partially or completely substituted with halogen atoms (F, Cl, Br, or I). Non-limiting examples of lower alkoxy groups include methoxy, ethoxy, n-propoxy, 1-methylethoxy (isopropoxy), n-butoxy, n-pentoxy, t-butoxy, and the like. Preferably, a lower alkyloxy group comprises 1-10 carbon atoms. More preferably, a lower alkyloxy group comprises 1-5 carbon atoms.

The term “cycloalkyl” or “cycloalkyl group” means a stable aliphatic saturated 3- to 15-membered monocyclic or polycyclic monovalent radical consisting solely of carbon and hydrogen atoms which may comprise one or more fused or bridged ring(s), preferably a 3- to 7-membered monocyclic rings. Other exemplary embodiments of cycloalkyl groups include 7- to 10-membered bicyclic rings. Unless otherwise specified, the cycloalkyl ring may be attached at any carbon atom which results in a stable structure and, if substituted, may be substituted at any suitable carbon atom which results in a stable structure. Exemplary cycloalkyl groups include cyclopropyl, cyclobutyl, cyclopentyl, cyclohexyl, cycloheptyl, cyclooctyl, cyclononyl, cyclodecyl, norbornyl, adamantyl, tetrahydronaphthyl (tetralin), 1-decalinyl, bicyclo[2.2.2]octanyl, 1-methylcyclopropyl, 2-methylcyclopentyl, 2-methylcyclooctyl, and the like.

As used herein, the term “aryl” or “aryl group” means an aromatic carbocyclic monovalent or divalent radical. In some embodiments, the aryl group has a number of carbon atoms from 5 to 24 and has a single ring (e.g., phenyl or phenylene), multiple condensed rings (e.g., naphthyl or anthranyl), or multiple bridged rings (e.g., biphenyl). Unless otherwise specified, the aryl ring may be attached at any suitable carbon atom which results in a stable structure and, if substituted, may be substituted at any suitable carbon atom which results in a stable structure. Non-limiting examples of aryl groups include phenyl, naphthyl, anthryl, phenanthryl, indanyl, indenyl, biphenyl, and the like. It may be abbreviated as “Ar”. Preferably, an aryl group comprises 5-14 carbon atoms. More preferably, an aryl group comprises 5-10 carbon atoms.

The term “heteroaryl” or “heteroaryl group” means a stable aromatic monocyclic or polycyclic monovalent or divalent radical, which may comprise one or more fused or bridged ring(s). In some embodiments, the heteroaryl group has 5-24 members, preferably a 5- to 7-membered monocyclic or 7- to 10-membered bicyclic radical. The heteroaryl group can have from one to four heteroatoms in the ring(s) independently selected from nitrogen, oxygen, and sulfur, wherein any sulfur heteroatoms may optionally be oxidized and any nitrogen heteroatom may optionally be oxidized or be quaternized. Unless otherwise specified, the heteroaryl ring may be attached at any suitable heteroatom or carbon atom which results in a stable structure and, if substituted, may be substituted at any suitable heteroatom or carbon atom which results in a stable structure. Non-limiting examples of heteroaryls include furanyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, triazinyl, indolizinyl, azaindolizinyl, indolyl, azaindolyl, diazaindolyl, dihydroindolyl, dihydroazaindoyl, isoindolyl, azaisoindolyl, benzofuranyl, furanopyridinyl, furanopyrimidinyl, furanopyrazinyl, furanopyridazinyl, dihydrobenzofuranyl, dihydrofuranopyridinyl, dihydrofuranopyrimidinyl, benzothienyl, thienopyridinyl, thienopyrimidinyl, thienopyrazinyl, thienopyridazinyl, dihydrobenzothienyl, dihydrothienopyridinyl, dihydrothienopyrimidinyl, indazolyl, azaindazolyl, diazaindazolyl, benzimidazolyl, imidazopyridinyl, benzthiazolyl, thiazolopyridinyl, thiazolopyrimidinyl, benzoxazolyl, benzoxazinyl, benzoxazinonyl, oxazolopyridinyl, oxazolopyrimidinyl, benzisoxazolyl, purinyl, chromanyl, azachromanyl, quinolizinyl, quinolinyl, dihydroquinolinyl, tetrahydroquinolinyl, isoquinolinyl, dihydroisoquinolinyl, tetrahydroisoquinolinyl, cinnolinyl, azacinnolinyl, phthalazinyl, azaphthalazinyl, quinazolinyl, azaquinazolinyl, quinoxalinyl, azaquinoxalinyl, naphthyridinyl, dihydronaphthyridinyl, tetrahydronaphthyridinyl, pteridinyl, carbazolyl, acridinyl, phenazinyl, phenothiazinyl, and phenoxazinyl, and the like.

Glucocorticoids (“GCs”) are among the most potent drugs used for the treatment of allergic and chronic inflammatory diseases or of inflammation resulting from infections. However, as mentioned above, long-term treatment with GCs is often associated with numerous adverse side effects, such as diabetes, osteoporosis, hypertension, glaucoma, or cataract. These side effects, like other physiological manifestations, are results of aberrant expression of genes responsible for such diseases. Research in the last decade has provided important insights into the molecular basis of GC-mediated actions on the expression of GC-responsive genes. GCs exert most of their genomic effects by binding to the cytoplasmic GC receptor (“GR”). The binding of GC to GR induces the translocation of the GC-GR complex to the cell nucleus where it modulates gene transcription either by a positive (transactivation) or negative (transrepression) mode of regulation. There has been growing evidence that both beneficial and undesirable effects of GC treatment are the results of undifferentiated levels of expression of these two mechanisms; in other words, they proceed at similar levels of effectiveness. Although it has not yet been possible to ascertain the most critical aspects of action of GCs in chronic inflammatory diseases, there has been evidence that it is likely that the inhibitory effects of GCs on cytokine synthesis are of particular importance. GCs inhibit the transcription, through the transrepression mechanism, of several cytokines that are relevant in inflammatory diseases, including IL-1β (interleukin-1β), IL-2, IL-3, IL-6, IL-11, TNF-α (tumor necrosis factor-α), GM-CSF (granulocyte-macrophage colony-stimulating factor), and chemokines that attract inflammatory cells to the site of inflammation, including IL-8, RANTES, MCP-1 (monocyte chemotactic protein-1), MCP-3, MCP-4, MIP-1α (macrophage-inflammatory protein-1α), and eotaxin. P. J. Barnes, Clin. Sci., Vol. 94, 557-572 (1998). On the other hand, there is persuasive evidence that the synthesis of IκB kinases, which are proteins having inhibitory effects on the NF-κB proinflammatory transcription factors, is increased by GCs. These proinflammatory transcription factors regulate the expression of genes that code for many inflammatory proteins, such as cytokines, inflammatory enzymes, adhesion molecules, and inflammatory receptors. S. Wissink et al., Mol. Endocrinol., Vol. 12, No. 3, 354-363 (1998); P. J. Barnes and M. Karin, New Engl. J. Med., Vol. 336, 1066-1077 (1997). Thus, both the transrepression and transactivation functions of GCs directed to different genes produce the beneficial effect of inflammatory inhibition. On the other hand, steroid-induced diabetes and glaucoma appear to be produced by the transactivation action of GCs on genes responsible for these diseases. H. Schäcke et al., Pharmacol. Ther., Vol. 96, 23-43 (2002). Thus, while the transactivation of certain genes by GCs produces beneficial effects, the transactivation of other genes by the same GCs can produce undesired side effects. Therefore, it is very desirable to provide pharmaceutical compounds, compositions, and methods for modulating inflammation without the undesired side effects of GC therapy.

In general, the present invention provides compositions and methods for modulating inflammation using fluoroquinolones.

In one aspect, the present invention provides compositions and methods for modulating ocular or ophthalmic inflammation using a novel fluoroquinolone.

In another aspect, such inflammation is anterior uveitis or vernal keratoconjunctivitis.

In still another aspect, such inflammation is intermediate uveitis, posterior uveitis, panuveitis, or secondary uveitis.

In still another aspect, such inflammation is acute anterior uveitis.

In another aspect, the present invention provides compositions comprising and methods for modulating ocular or ophthalmic inflammation using a fluoroquinolone having Formula 1 or a salt thereof.

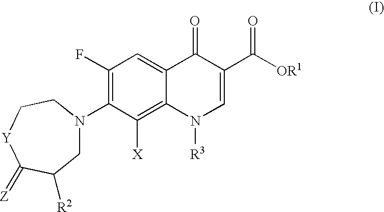

wherein R¹ is selected from the group consisting of hydrogen, unsubstituted lower alkyl groups, substituted lower alkyl groups, cycloalkyl groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, and groups that can be hydrolyzed in living bodies; R² is selected from the group consisting of hydrogen, unsubstituted amino group, and amino groups substituted with one or two lower alkyl groups; R³ is selected from the group consisting of hydrogen, unsubstituted lower alkyl groups, substituted lower alkyl groups, cycloalkyl groups, unsubstituted lower alkoxy groups, substituted lower alkoxy groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, unsubstituted C₅-C₂₄ aryloxy groups, substituted C₅-C₂₄ aryloxy groups, unsubstituted C₅-C₂₄ heteroaryloxy groups, substituted C₅-C₂₄ heteroaryloxy groups, and groups that can be hydrolyzed in living bodies; X is selected from the group consisting of halogen atoms; Y is selected from the group consisting of CH₂, O, S, SO, SO₂, and NR⁴, wherein R⁴ is selected from the group consisting of hydrogen, unsubstituted lower alkyl groups, substituted lower alkyl groups, and cycloalkyl groups; and Z is selected from the group consisting of oxygen and two hydrogen atoms.

In still another aspect, a composition of the present invention for modulating an inflammation comprises a member of a family of fluoroquinolones having Formula II or salts thereof,

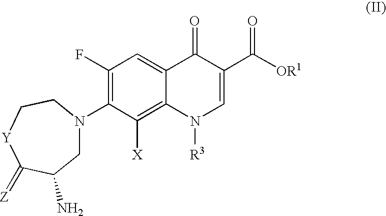

wherein R¹, R³, X, Y, and Z have the meanings as disclosed above; and a method of the present invention for modulating an inflammation uses such a fluoroquinolone.

In still another aspect, the present invention provides compositions comprising, and methods for treating, controlling, reducing, or ameliorating an ocular or ophthalmic infection and its inflammatory sequelae in a subject using, a fluoroquinolone having Formula I or II, or a salt thereof.

In one aspect, R¹ is selected from the group consisting of hydrogen, C₁-C₅ (or alternatively, C₁-C₃) substituted and unsubstituted alkyl groups, C₃-C₁₀ (or alternatively, C₃-C₅) cycloalkyl groups, C₅-C₁₄ (or alternatively, C₆-C₁₄, or C₅-C₁₀, or C₆-C₁₀) substituted and unsubstituted aryl groups, C₅-C₁₄ (or alternatively, C₆-C₁₄, or C₅-C₁₀, or C₆-C₁₀) substituted and unsubstituted heteroaryl groups, and groups that can be hydrolyzed in living bodies. In one embodiment, R¹ is selected from the group consisting of C₁-C₅ (or alternatively, C₁-C₃) substituted and unsubstituted alkyl groups.

In another aspect, R² is selected from the group consisting of unsubstituted amino group and amino groups substituted with one or two C₁-C₅ (or alternatively, C₁-C₃) alkyl groups.

In still another aspect, R³ is selected from the group consisting of hydrogen, C₁-C₅ (or alternatively, C₁-C₃) substituted and unsubstituted alkyl groups, C₃-C₁₀ (or alternatively, C₃-C₅) cycloalkyl groups, C₁-C₅ (or alternatively, C₁-C₃) substituted and unsubstituted alkoxy groups, C₅-C₁₄ (or alternatively, C₆-C₁₄, or C₅-C₁₀, or C₆-C₁₀) substituted and unsubstituted aryl groups, C₅-C₁₄ (or alternatively, C₆-C₁₄, or C₅-C₁₀, or C₆-C₁₀) substituted and unsubstituted heteroaryl groups, and C₅-C₁₄ (or alternatively, C₆-C₁₄, or C₅-C₁₀, or C₆-C₁₀) substituted and unsubstituted aryloxy groups. In one embodiment, R³ is selected from the group consisting of C₃-C₁₀ (or alternatively, C₃-C₅) cycloalkyl groups.

In yet another aspect, X is selected from the group consisting of Cl, F, and Br. In one embodiment, X is Cl. In another embodiment, X is F.

In a further aspect, Y is CH₂. In still another aspect, Z comprises two hydrogen atoms.

In still another aspect, Y is NH, Z is O, and X is Cl.

In another aspect, a composition of the present invention further comprises a pharmaceutically acceptable carrier.

Some non-limiting members of the family of compounds having Formula I are shown in Table 1. Other compounds of the family not listed in Table 1 are also suitable in selected situations.

TABLE 1
Some Selected Fluoroquinolones

| Com- | | | | | | |
|---|---|---|---|---|---|---|
| pound | R¹ | R² | R³ | X | Y | Z |
| 1 | H | H | CH₃ | Cl | CH₂ | 2H |
| 2 | H | NH₂ | CH₃ | Cl | CH₂ | 2H |
| 3 | H | NH₂ | cyclopropyl | Cl | CH₂ | 2H |
| 4 | H | NH(CH₃) | cyclopropyl | Cl | CH₂ | 2H |
| 5 | H | N(CH₃)₂ | cyclopropyl | Cl | CH₂ | 2H |
| 6 | CH₃ | NH₂ | cyclopropyl | Cl | CH₂ | 2H |
| 7 | C₂H₅ | NH₂ | cyclopropyl | Cl | CH₂ | 2H |
| 8 | H | NH₂ | cyclopropyl | F | CH₂ | 2H |
| 9 | H | NH₂ | cyclopropyl | Br | CH₂ | 2H |
| 10 | H | NH(C₂H₅) | cyclopropyl | Cl | CH₂ | 2H |
| 11 | H | NH(C₃H₇) | cyclopropyl | F | CH₂ | 2H |
| 12 | H | NH₂ | cyclopentyl | Cl | CH₂ | 2H |
| 13 | H | NH₂ | cyclopropyl | Cl | CH₂ | O |
| 14 | H | NH₂ | cyclopropyl | F | CH₂ | O |
| 15 | H | NH₂ | cyclopropyl | Br | CH₂ | O |
| 16 | H | NH₂ | cyclopropyl | Cl | CH(C₂H₅) | O |
| 17 | CH₃ | NH₂ | cyclopropyl | Cl | CH₂ | O |
| 18 | CH₃ | NH(CH₃) | cyclopropyl | Cl | CH₂ | O |
| 19 | CH₃ | N(CH₃)₂ | cyclopropyl | Cl | CH₂ | O |
| 20 | CH₃ | NH(C₃H₇) | cyclopropyl | Cl | CH₂ | O |
| 21 | CH₃ | NH(C₂H₅) | cyclopropyl | Cl | CH₂ | O |
| 22 | CH₃ | N(CH₃)(C₂H₅) | cyclopropyl | Cl | CH₂ | O |
| 23 | H | NH₂ | cyclopropyl | Cl | NH | O |
| 24 | CH₃ | NH(CH₃) | cyclopropyl | Cl | NH | O |
| 25 | H | 2H | cyclopropyl | Cl | NH | O |

In one embodiment, the fluoroquinolone carboxylic acid included in a composition and used in a method of the present invention has Formula III.

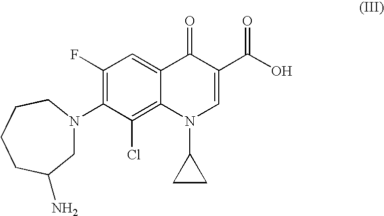

In another embodiment, the fluoroquinolone carboxylic acid included in a composition and used in a method of the present invention has Formula IV, V, or VI.

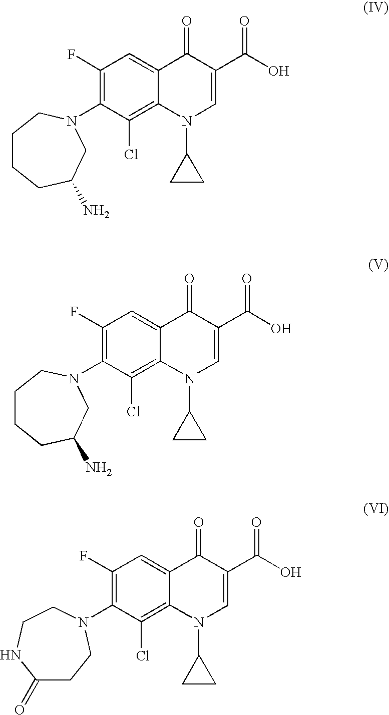

In still other embodiments, the fluoroquinolone carboxylic acid included in a composition and used in a method of the present invention has Formula VII or VIII.

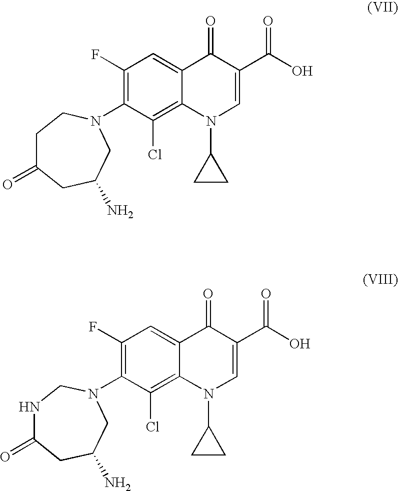

In still another aspect, a composition of the present invention comprises an enantiomer of one of the compounds having Formula I, II, or III, and a method of the present invention uses one or more such compounds.

In still another aspect, a composition of the present invention comprises a mixture of enantiomers of one of the compounds having Formula I, II, or III, and a method of the present invention uses such a mixture.

A fluoroquinolone disclosed herein can be produced by a method disclosed in U.S. Pat. Nos. 5,447,926 and 5,385,900, which are incorporated herein by reference.

In yet another aspect, the present invention provides a method for modulating an inflammation in a subject. The method comprises administering into the subject an effective amount of the fluoroquinolone having Formula I, II, III, IV, V, VI, VII, or VIII, or a salt thereof to modulate the inflammation.

In still another aspect, the present invention provides a method for treating, controlling, reducing, or ameliorating an infection and its inflammatory sequelae in a subject. The method comprises administering into the subject an effective amount of a fluoroquinolone having Formula I, II, III, IV, V, VI, VII, or VIII, or a salt thereof to treat, control, reduce, or ameliorate such an infection and its inflammatory sequelae.

In yet another aspect, such an infection is caused by bacteria, viruses, fungi, protozoans, or combinations thereof.

In still another aspect, such an infection is an ocular or ophthalmic infection.

In still another aspect, such an ocular or ophthalmic infection is selected from the group consisting of blepharitis, conjunctivitis, keratitis, trachoma, and combinations thereof. In one embodiment, such an infection is selected from the group consisting of anterior blepharitis, posterior blepharitis, herpes simplex keratitis, herpes zoster keratitis, bacterial keratitis, fungal keratitis (such as fusarium keratitis), acanthamoeba keratitis, cytomegalovirus retinitis, toxoplasma retinitis, herpes zoster conjunctivitis, bacterial conjunctivitis, bacterial infection of aqueous and vitreous humours, endophthalmitis, panophthalmitis, trachoma, and combinations thereof.

In yet another aspect, the present invention provides a composition and a method for modulating an inflammatory response accompanying corneal infiltrates, wherein such a composition comprises one of the fluoroquinolones having Formula I, II, III, IV, V, VI, VII, or VIII, and such a method employs such a composition. The term “corneal infiltrates” refers to inflammatory cells of the immune system that enter the cornea in response to stressors such as toxins, ocular irritants, or other materials foreign to the ocular environment. Corneal infiltrates are typically composed of polymorphonulclear leukocytes (neutrophils), but may also contain lymphocytes and macrophages. Infiltrating cells may migrate from the limbal vasculature or from the tear film in response to local tissue damage and chemotactic factors, induced by antigens and toxins from the environment including components from microbial organisms. In one embodiment, the corneal infiltrates are contact lens-associated corneal infiltrates (“CLACI”). Any one or a combination of multiple mechanical, hypoxic, toxic, or irritating stimuli associated with contact lens use can induce proinflammatory responses that lead to infiltration of inflammatory cells into the cornea. In one aspect, corneal infiltrates may be associated with the presence of microbes at the ocular surface. These microbes may not directly cause tissue damage (infection) but can elicit an innate immune response by release of cellular components such as endotoxin, cell wall materials, or nucleic acids. M. W. Robboy et al., Eye & Contact Lens, Vol. 29, No. 3, 146 (2003).

In yet another aspect, the present invention provides compositions and methods for treating, controlling, reducing, ameliorating, or alleviating an inflammation or an infection and its inflammatory sequelae in a subject, which compositions and methods cause a lower level of at least an adverse side effect than compositions comprising at least a prior-art glucocorticoid used to treat, control, reduce, or ameliorate the same conditions (said inflammation or infection and its inflammatory sequelae).

In one aspect, a level of said at least an adverse side effect is determined in vivo or in vitro. For example, a level of said at least an adverse side effect is determined in vitro by performing a cell culture and determining the level of a biomarker associated with said side effect. Such biomarkers can include proteins (e.g., enzymes), lipids, sugars, and derivatives thereof that participate in, or are the products of, the biochemical cascade resulting in the adverse side effect. Representative in vitro testing methods are further disclosed hereinbelow.

In still another aspect, said at least an adverse side effect is selected from the group consisting of glaucoma, cataract, hypertension, hyperglycemia, hyperlipidemia (increased levels of triglycerides), and hypercholesterolemia (increased levels of cholesterol).

In another embodiment, a level of said at least an adverse side effect is determined at about one day after said composition is first administered to, and are present in, said subject. In another embodiment, a level of said at least an adverse side effect is determined about 14 days after said composition is first administered to, and are present in, said subject. In still another embodiment, a level of said at least an adverse side effect is determined about 30 days after said composition is first administered to, and are present in, said subject. Alternatively, a level of said at least an adverse side effect is determined about 2, 3, 4, 5, or 6 months after said compounds or compositions are first administered to, and are present in, said subject.

In another aspect, said at least a prior-art glucocorticoid used to treat, control, reduce, or ameliorate the same conditions is administered to said subject at a dose and a frequency sufficient to produce an equivalent beneficial effect on said condition to a composition of the present invention after about the same elapsed time.

In still another aspect, said at least a prior-art glucocorticoid is selected from the group consisting of 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, their physiologically acceptable salts, combinations thereof, and mixtures thereof. In one embodiment, said at least a prior-art glucocorticoid is selected from the group consisting of dexamethasone, prednisone, prednisolone, methylprednisolone, medrysone, triamcinolone, loteprednol etabonate, physiologically acceptable salts thereof, combinations thereof, and mixtures thereof. In another embodiment, said at least a prior-art glucocorticoid is acceptable for ophthalmic uses.

### Testing for Inhibition of LPS-Induced Cytokine Expression in Human THP-1 Monocytes by Compound Having Formula IV and Moxifloxacin

### Experimental Method

Human THP-1 monocytes (ATCC TIB 202) were purchased from American Type Culture Collection (Manassas, Va.) and maintained in RPMI 1640 medium (Invitrogen, Carlsbad, Calif.) supplemented with 10% fetal bovine serum (“FBS”, Invitrogen, Carlsbad, Calif.), 100 U/mL of penicillin (Invitrogen, Carlsbad, Calif.), and 100 μg/mL of streptomycin (Invitrogen, Carlsbad, Calif.) at 37° C. in a humidified incubator with 5% CO₂. THP-1 cells were pre-cultured in RPMI 1640 medium containing 10% dialyzed serum for 24 h. Cells were seeded in 24-well plates in RPMI 1640 medium containing 2% dialyzed serum (purchased from Hyclone, Loga, Utah) and treated with vehicle (DMSO, dimeththyl sulfoxide), 10 μg/mL LPS (Sigma Aldrich, St. Louis, Mo.), 0.1, 1, 10 or 30 μg/mL moxifloxacin (Neuland laboratories, Hyderabad, India), 0.1, 1, 10 or 30 μg/mL compound having Formula IV (Bausch & Lomb Incorporated, Rochester, New York), 10 μg/ml LPS+0.1, 1, 10 or 30 μg/mL moxifloxacin, or 10 μg/ml LPS+0.1, 1, 10 or 30 μg/mL compound having Formula IV for 18 hours. Each treatment was performed in triplicate.

### Multiplex Luminex

Samples were analyzed using multiplex bead technology, which utilizes microspheres as the solid support for immunoassays and allows the analysis of all cytokines from each sample (D. A. Vignali, J. Immunol. Methods, Vol. 243, 243-255 (2000)). Sixteen cytokines were measured according to the manufacturer's instructions. Briefly, 50 μL of medium samples were incubated with antibody-coated capture beads overnight at 4° C. Washed beads were further incubated with biotin-labelled anti-human cytokine antibodies for 2 h at room temperature followed by incubation with streptavidin-phycoerythrin for 30 min. Samples were analyzed using Luminex 200™ (Luminex, Austin, Tex.) and Beadview software v1.0 (Upstate Cell Signaling Solutions, Temecula, Calif.). Standard curves of known concentrations of recombinant human cytokines were used to convert fluorescence units (median fluorescence intensity) to cytokine concentration in pg/mL. Only the linear portions of the standard curves were used to quantify cytokine concentrations, and in instances where the fluorescence reading exceeded the linear range of the standard curve, an appropriate dilution was performed to ensure that the concentration was in the linear portion of the curve.

### Cellular Metabolic Function

Cellular metabolic competence was determined by an AlamarBlue assay (J. O'Brien et al., FEBS J., Vol. 267, 5421-5426 (2000)). Briefly, after removal of medium, cells were incubated with 1:10 diluted AlamarBlue solution (Biosource, Camarillo, Calif.) for 3 hours at 37° C. in a humidified incubator with 5% CO₂. The plate was read fluorometrically by excitation at 530-560 nm and emission at 590 nm. Relative fluorescence units (“RFU”) were used to determine cell viability

### Data Analysis and Statistics

All cytokine concentrations (pg/mL) were expressed as mean ±standard deviation. Statistical analysis comparing effects of treatment across groups was performed using a one-way ANOVA with a Dunnett's post-hoc comparison test using either vehicle control or LPS treatment as references. For all assays, p≦0.05 was predetermined as the criterion of statistical significance.

### Results

In no instance did any of the treatments produce a statistically significant effect on cellular metabolic activity as measured by the AlamarBlue assay (data not shown). The overall results from the studies determining cytokine levels in the culture medium from these various treatment groups are summarized in Table 2. Substantial levels of 14 out of the 16 cytokines in the assay were detectable in culture media from THP-1 monocytes, with all cytokines except EGF and IL-7 affected. Exposure of THP-1 monocytes to 10 μg/mL of LPS for 18 hours resulted in a significant increase of 13 out of the 14 detectable cytokines; the amount of VEGF in THP-1 monocyte culture medium also increased, but the increase did not attain statistical significance.

TABLE 2
Summary of inhibition of LPS-stimulated cytokine production by
moxifloxacin and Compound Having Formula IV in human
THP-1 monocytes

| | | | | | Inhibited by | | | |
|---|---|---|---|---|---|---|---|---|
| | Inhibited by | | | | Compound Having | | | |
| | Moxifloxacin at μg/mL | | | | Formula IV at μg/mL | | | |
| Cytokine | 0.1 | 1 | 10 | 30 | 0.1 | 1 | 10 | 30 |
| Fractalkine | | | | | | | | |
| G-CSF | | | | X | | X | X | X |
| GM-CSF | | | | X | | | | X |
| IL-12p40 | | X | X | X | | | | X |
| IL-1α | | | | X | X | X | X | X |
| IL-1β | | | | X | | | | X |
| IL-1ra | | | X | X | | X | X | X |
| IL-6 | | | X | | | X | X | X |
| IL-8 | | | | X | | | | X |
| IP-10 | | | | X | | | | X |
| MCP-1 | | | | | | | | X |
| MIP-1α | | | | X | | | | X |
| RANTES | | | | | | | | |
| VEGF | | | | X | | | X | X |

Note:
“X” signifies significant inhibition at a particular concentration.

Both moxifloxacin and compound having Formula IV significantly inhibited LPS-induced cytokine production in THP-1 monocytes. For moxifloxacin, a significant inhibitory effect was observed at 1 μg/ml for IL-12p40, at 10 μg/ml for IL-1ra and IL-6, and at 30 μg/ml for G-CSF, GM-CSF, IL-1α, IL-1β, IL-8, IP-10, and MIP-1α (Table 1). For compound having Formula IV, a significant inhibitory effect was observed at 0.1 μg/ml for IL-1α, at 1 μg/ml for G-CSF, IL-1ra and IL-6, and at 30 μg/ml for GM-CSF, IL-12p40, IL-1β, IL-1ra, IL-8, IP-10, MCP-1 and MIP-1a (Table 2). Neither moxifloxacin nor compound having Formula IV altered LPS-stimulated production of RANTES or fractalkine.

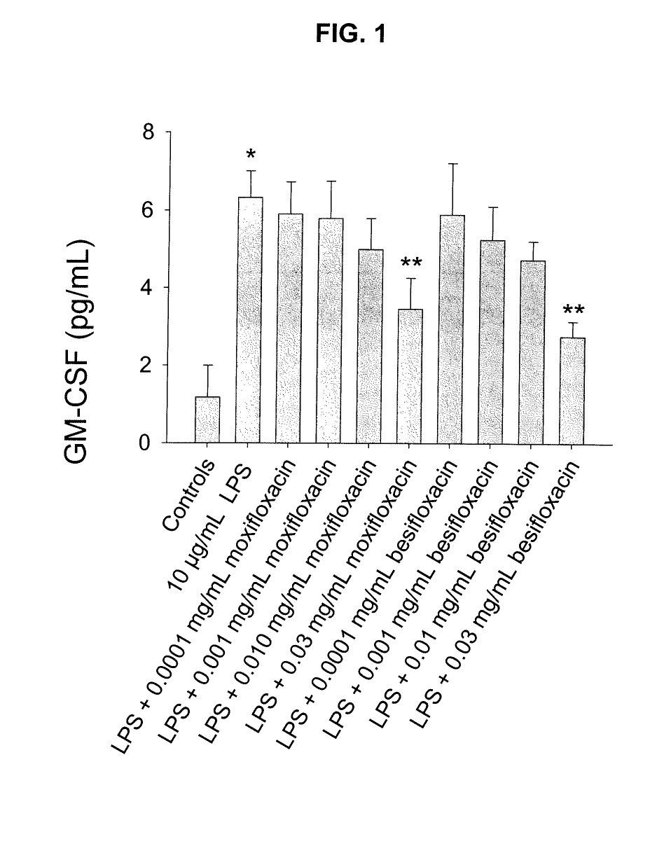

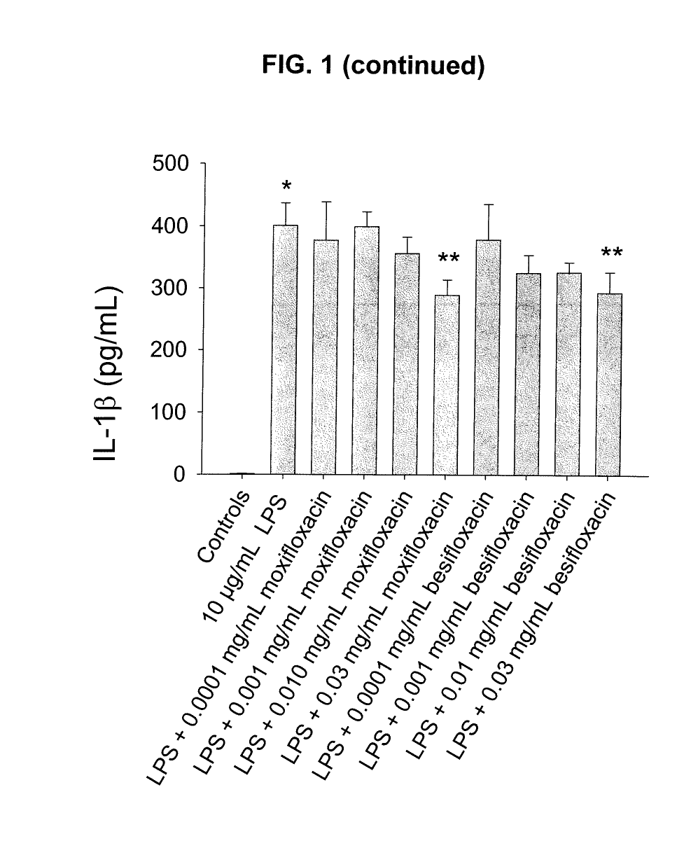

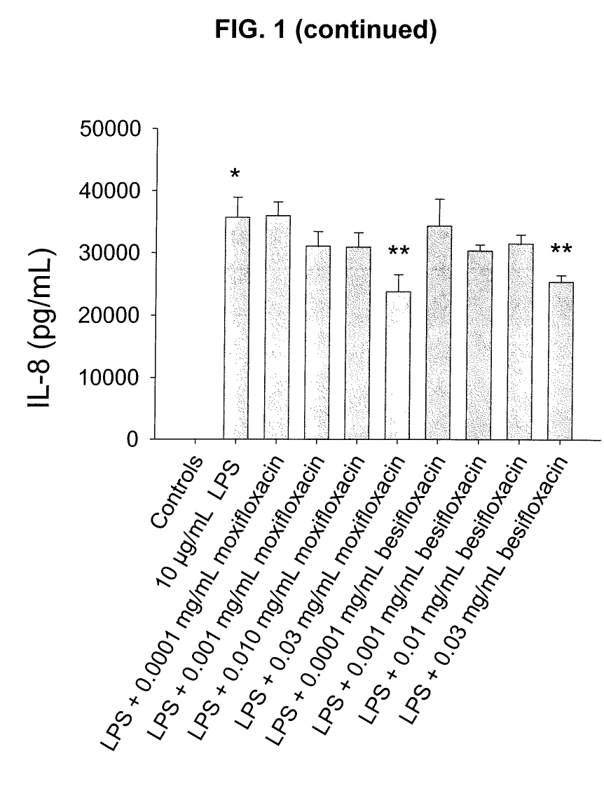

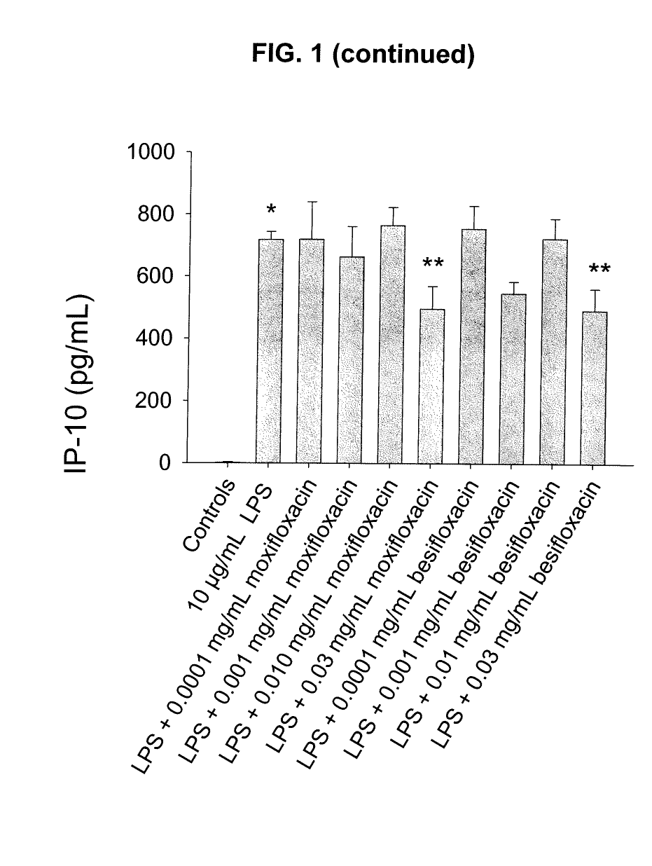

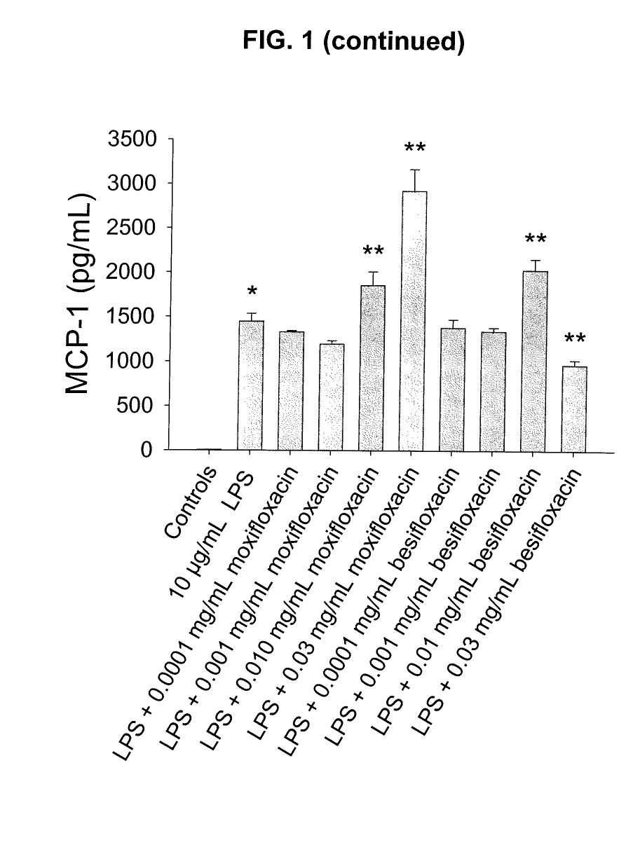

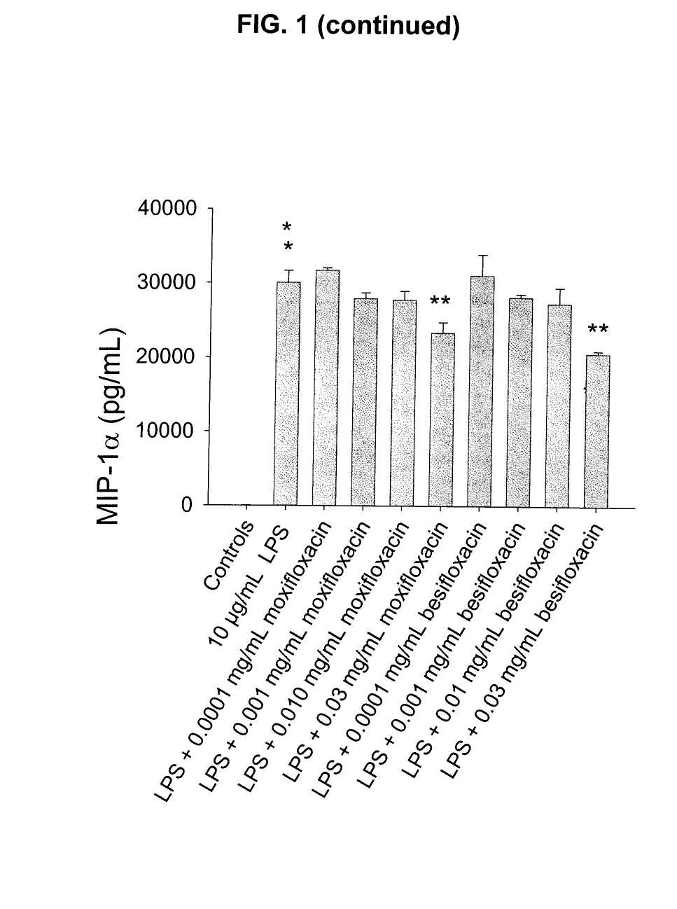

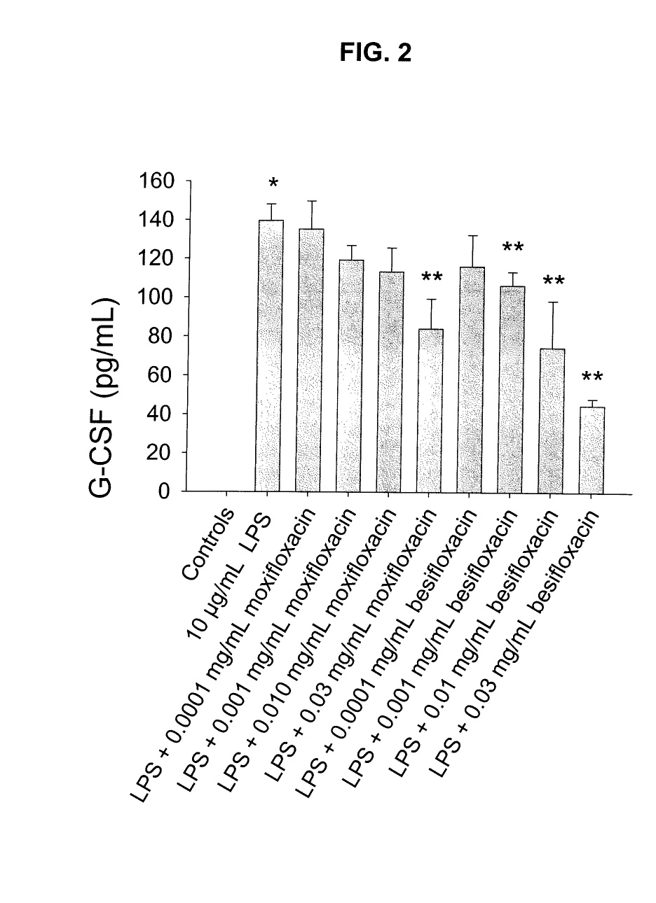

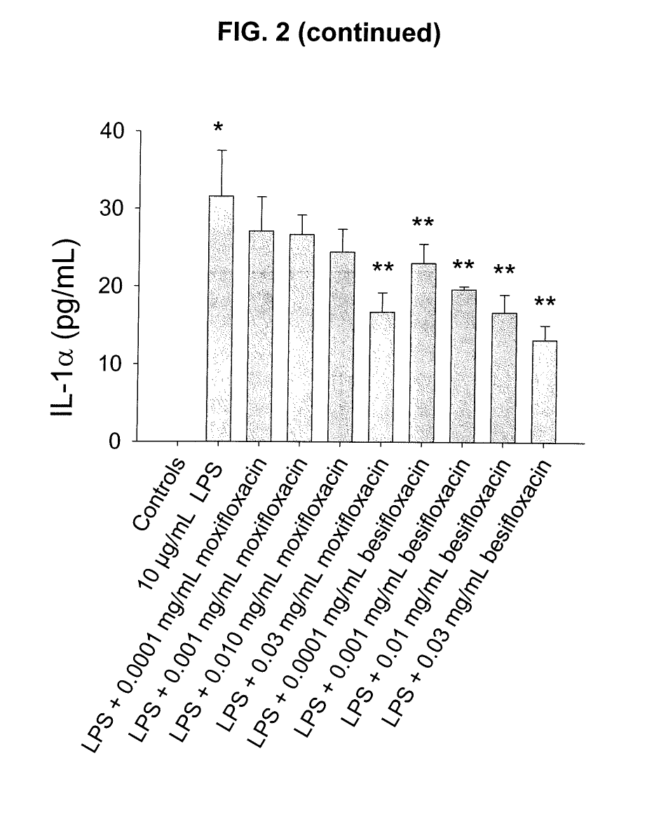

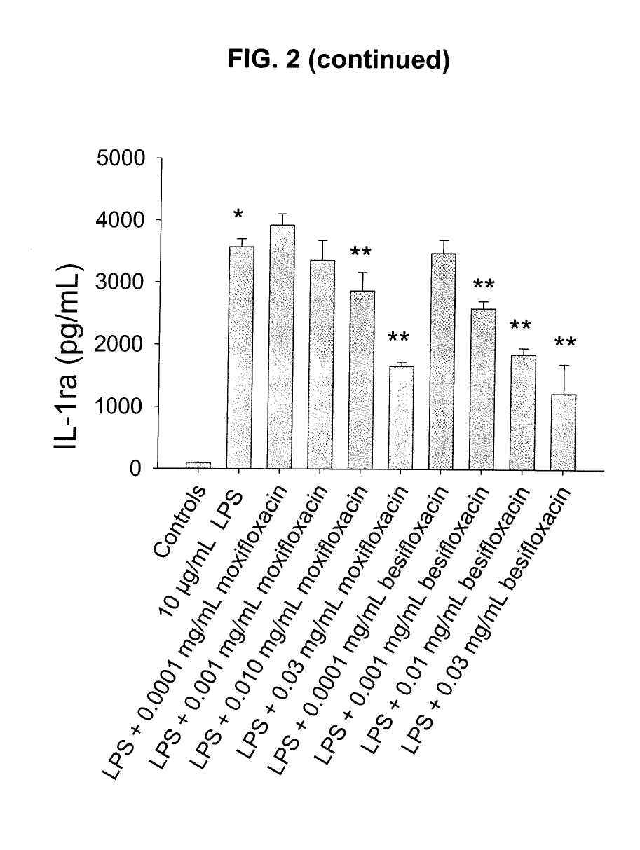

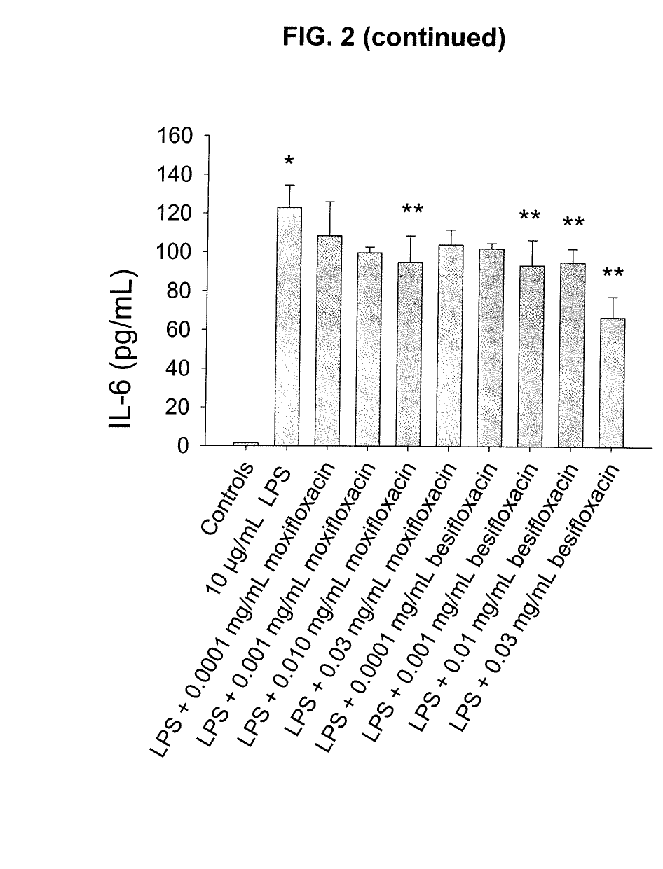

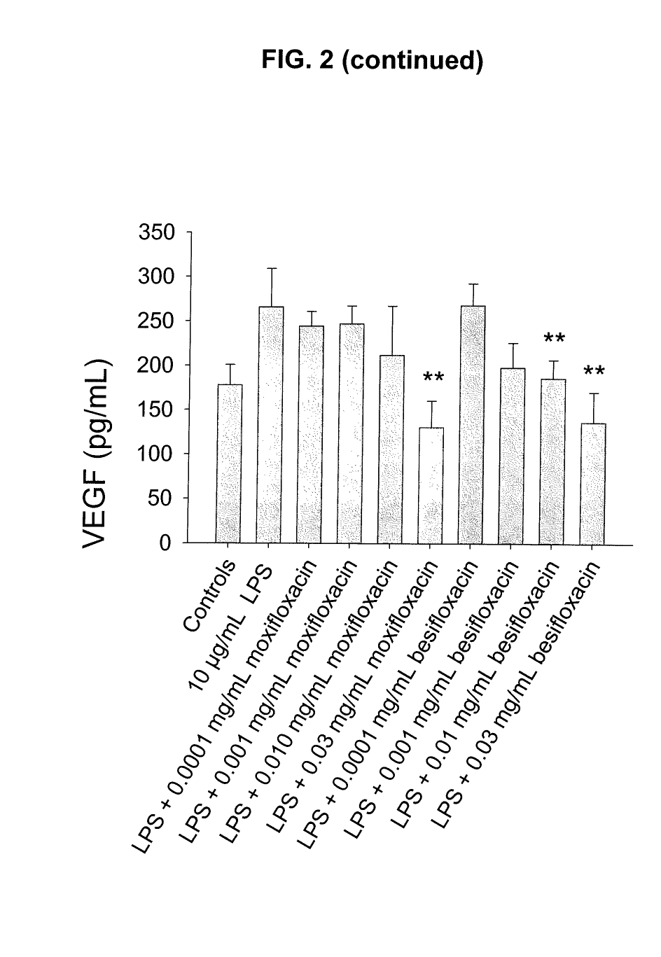

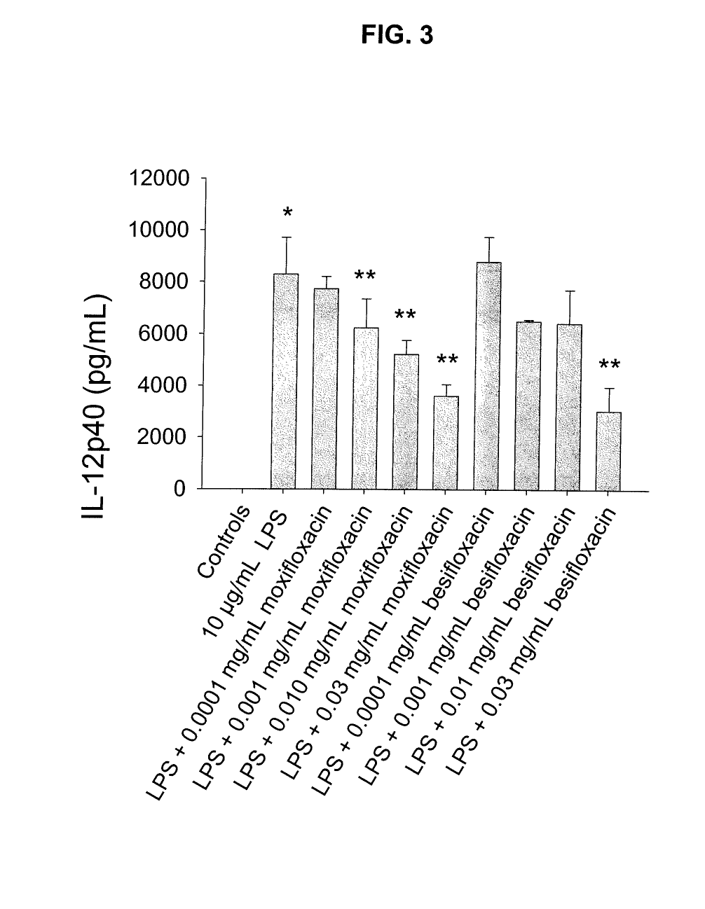

The cytokines detected in this study were divisible into four different response groups. The first group includes those cytokines for which these fluoroquinolones had no significant efficacy (RANTES and fractalkine). The second group of cytokines includes GM-CSF, IL-1β, IL-8, IP-10, MCP-1, and MIP-1α. For these cytokines, both moxifloxacin and compound having Formula IV (labeled as BOL-303224-A in the figures) had comparable effects after LPS stimulation (FIG. 1). The third group of cytokines, including G-CSF, IL-1α, IL-1ra, IL-6, and VEGF are those for which compound having Formula IV demonstrated better potency than moxifloxacin (FIG. 2). Finally, the fourth group of cytokines are those for which moxifloxacin was more potent than compound having Formula IV, and consists of only IL-12p40 (FIG. 3).

With the compound having Formula IV, significant cytokine inhibitory effects were observed at very low concentrations. For example, a significant inhibitory effect of compound having Formula IV was seen at as low as 100 ng/mL on IL-1α, and at 1000 ng/mL on G-CSF, IL-1ra, and IL-6. These concentrations are well below predicted ocular concentrations following topical administration (K. W. Ward et al., J. Ocul. Pharmacol. Ther., Vol. 23, 243-256 (2007)). Therefore, clinical benefits resulting from this cytokine inhibition profile can be obtained.

A fluoroquinolone compound disclosed herein can be formulated into a pharmaceutical composition for topical, oral, subcutaneous, or systemic administration for the modulation of inflammation or the treatment, reduction, or amelioration of an infection and its inflammatory sequelae. Such a composition comprises a fluoroquinolone compound having Formula I, II, III, IV, V, VI, VII, or VIII or a salt thereof and a pharmaceutically acceptable carrier for the administration, as can be determined by a person having skill in the art of pharmaceutical formulation. For example, various pharmaceutically acceptable carriers known in the art can be used to formulate a solution, emulsion, suspension, dispersion, ointment, gel, capsule, or tablet. A fluoroquinolone compound having Formula I, II, III, IV, V, VI, VII, or VIII or a salt thereof is particularly suitable for a treatment, reduction, amelioration, or prevention of infections of the ear, eye, or a portion of the upper respiratory tract, caused by microorganisms. Such a fluoroquinolone or a salt thereof is formulated into a solution, ointment, suspension, dispersion, or gel.

In one embodiment, a topical composition of the present invention comprises an aqueous solution or suspension. Typically, purified or deionized water is used. The pH of the composition is adjusted by adding any physiologically acceptable pH adjusting acids, bases, or buffers to within the range of about 3 to about 8.5 (or alternatively, or from about 4 to about 7.5, or from about 4 to about 6.5, or from about 5 to about 6.5). Examples of acids include acetic, boric, citric, lactic, phosphoric, hydrochloric, and the like, and examples of bases include sodium hydroxide, potassium hydroxide, tromethamine, THAM (trishydroxymethylaminomethane), and the like. Salts and buffers include citrate/dextrose, sodium bicarbonate, ammonium chloride and mixtures of the aforementioned acids and bases. pH buffers are introduced into the composition to maintain a stable pH and to improve product tolerance by the user. In some embodiments, the pH is in the range from about 4 to about 7.5. Biological buffers for various pHs are available, for example, from Sigma-Aldrich. A composition of the present invention can have a viscosity in the range from about 5 to about 100,000 centipoise (“cp”) or mPa·s (or alternatively, from about 10 to about 50,000, or from about 10 to about 20,000, or from about 10 to about 10,000, or from about 10 to about 1,000, or from about 100 to about 10,000, or from about 100 to about 20,000, or from about 100 to about 50,000 or from about 500 to about 10,000, or from about 500 to about 20,000 cp).

In another embodiment, a topical composition of the present invention comprises an ointment, emulsion or cream (such as oil-in-water emulsion), or gel.

Ointments generally are prepared using either (1) an oleaginous base; i.e., one consisting of fixed oils or hydrocarbons, such as white petrolatum or mineral oil, or (2) an absorbent base; i.e., one consisting of an anhydrous substance or substances which can absorb water, for example anhydrous lanolin. Customarily, following formation of the base, whether oleaginous or absorbent, the active ingredient (compound) is added to an amount affording the desired concentration.

Creams are oil/water emulsions. They consist of an oil phase (internal phase), comprising typically fixed oils, hydrocarbons, and the like, such as waxes, petrolatum, mineral oil, and the like, and an aqueous phase (continuous phase), comprising water and any water-soluble substances, such as added salts. The two phases are stabilized by use of an emulsifying agent, for example, a surface active agent, such as sodium lauryl sulfate, hydrophilic colloids, such as acacia colloidal clays, veegum, and the like. Upon formation of the emulsion, the active ingredient (compound) customarily is added in an amount to achieve the desired concentration.

Gels comprise a base selected from an oleaginous base, water, or an emulsion-suspension base. To the base is added a gelling agent which forms a matrix in the base, increasing its viscosity. Examples of gelling agents are hydroxypropyl cellulose, acrylic acid polymers, and the like. Customarily, the active ingredient (compound) is added to the formulation at the desired concentration at a point preceding addition of the gelling agent.

The amount of a fluoroquinolone compound herein disclosed that is incorporated into a composition of the present invention is not critical; the concentration should be within a range sufficient to permit ready application of the formulation to the affected tissue area in an amount which will deliver the desired amount of compound to the desired treatment site and to provide the desired therapeutic effect. In some embodiments of the present invention, compositions comprise a fluoroquinolone in a concentration in a range from about 0.0001% to 10% by weight (or alternatively, from about 0.001% to about 5%, or from about 0.01% to about 5%, or from about 0.01% to about 2%, or from about 0.01% to about 1%, or from about 0.01% to about 0.7%, or from about 0.01% to about 0.5%, by weight).

Moreover, a topical composition of the present invention can contain one or more of the following: preservatives, surfactants, adjuvants including additional medicaments, antioxidants, tonicity adjusters, viscosity modifiers, and the like.

Preservatives may be used to inhibit microbial contamination of the product when it is dispensed in single or multidose containers, and can include: quaternary ammonium derivatives, (benzalkonium chloride, benzylammonium chloride, cetylmethyl ammonium bromide, cetylpyridinium chloride), benzethonium chloride, organomercury compounds (Thimerosal, phenylmercury acetate, phenylmercury nitrate), methyl and propyl p-hydroxy-benzoates, betaphenylethyl alcohol, benzyl alcohol, phenylethyl alcohol, phenoxyethanol, and mixtures thereof. These compounds are used at effective concentrations, typically from about 0.005% to about 5% (by weight), depending on the preservative or preservatives selected. The amount of the preservative used should be enough so that the solution is physically stable; i.e., a precipitate is not formed, and antibacterially effective.

The solubility of the components, including a fluoroquinolone having Formula I, II, III, IV, V, VI, VII, or VIII, of the present compositions may be enhanced by a surfactant or other appropriate co-solvent in the composition or solubility enhancing agents like cyclodextrins such as hydroxypropyl, hydroxyethyl, glucosyl, maltosyl and maltotriosyl derivatives of α-, β-, and γ-cyclodextrin. In one embodiment, the composition comprises 0.1% to 20% hydroxypropyl-β-cyclodextrin; alternatively, 1% to 15% (or 2% to 10%) hydroxypropyl-β-cyclodextrin. Co-solvents include polysorbates (for example, polysorbate 20, 60, and 80), polyoxyethylene/polyoxypropylene surfactants (e.g., Pluronic® F68, F84, F127, and P103), cyclodextrin, fatty-acid triglycerides, glycerol, polyethylene glycol, other solubility agents such as octoxynol 40 and tyloxapol, or other agents known to those skilled in the art and mixtures thereof. The amount of solubility enhancer used will depend on the amount of fluoroquinolone in the composition, with more solubility enhancer used for greater amounts of fluoroquinlones. Typically, solubility enhancers are employed at a level of from 0.01% to 20% (alternatively, 0.1% to 5%, or 0.1% to 2%) by weight depending on the ingredient.

The use of viscosity enhancing agents to provide the compositions of the invention with viscosities greater than the viscosity of simple aqueous solutions may be desirable to increase absorption of the active compounds by the target tissues or to increase the retention time therein. Such viscosity enhancing agents include, for example, polyvinyl alcohol, polyvinyl pyrrolidone, methyl cellulose, hydroxypropylmethyl cellulose, hydroxyethyl cellulose, carboxymethyl cellulose, hydroxypropyl cellulose or other agents known to those skilled in the art. Such agents are typically employed at a level of from 0.01% to 10% (alternatively, 0.1% to 5%, or 0.1% to 2%) by weight.

Suitable surfactants include polyvinyl pyrolidone, polyvinyl alcohol, polyethylene glycol, ethylene glycol, and propylene glycol. Other surfactants are polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). The surfactant helps a topical formulation to spread on the surface of narrow passages.

In one aspect, it may be desirable to include in a composition of the present invention at least another anti-inflammatory agent. Preferred anti-inflammatory agents include the well-known non-steroidal anti-inflammatory drugs (“NSAIDs”).

Non-limiting examples of the NSAIDs are: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lomoxicam, piroxicam, tenoxicam), ε-acetamidocaproic acid, S-(5′-adenosyl)-L-methionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof. In one embodiment, the NSAID is diclofenac, furbiprofen, or ketorolac.

Other non-steroidal anti-inflammatory agents include the cyclooxygenase type II selective inhibitors, such as celecoxib, and etodolac; PAF (platelet activating factor) antagonists, such as apafant, bepafant, minopafant, nupafant, and modipafant; PDE (phosphodiesterase) IV inhibitors, such as ariflo, torbafylline, rolipram, filaminast, piclamilast, cipamfylline, and roflumilast; inhibitors of cytokine production, such as inhibitors of the NF-κB transcription factor; or other anti-inflammatory agents known to those skilled in the art. In one embodiment, the non-steroidal anti-inflammatory agent is celecoxib.

The concentrations of the anti-inflammatory agents contained in the compositions of the present invention will vary based on the agent or agents selected and the type of inflammation being treated. The concentrations will be sufficient to reduce, treat, or prevent inflammation in the targeted tissues following application of a composition of the present invention to those tissues. Such concentrations are typically in the range from about 0.0001 to about 3% by weight (or alternatively, from about 0.01 to about 2%, or from about 0.05% to about 1%, or from about 0.01% to about 0.5%, by weight).

The following examples are provided to further illustrate non-limiting compositions of the present invention, and methods of preparing such composition, for the treatment, reduction, amelioration, or prevention of infections and inflammatory sequelae thereof.

### EXAMPLE 1

### Solution

| Ingredient | Amount (% by weight) |
|---|---|
| Compound having Formula IV | 0.2 |
| Hydroxypropylmethylcellulose (“HPMC”) | 0.5 |
| Benzakonium chloride (“BAK”) | 0.01 |
| Pluronic ® F127 | 0.1 |
| EDTA | 0.1 |
| NaCl | 0.25 |
| Phosphate buffer (0.05M, pH = 5.0) | q.s. to 100 |

An appropriate proportion (shown in the above table) of Pluronic® F127 is added to phosphate buffer in a sterilized stainless steel jacketed vessel equipped with a stirring mechanism, at a temperature in the range from 50 to 60° C. The resulting buffer solution is heated to 61 to 75° C. At a temperature of about 66° C., an appropriate amount of BAK is added to the buffer solution while mixing three to ten minutes. At a temperature of 75° C., an appropriate amount of the compound having Formula IV is added to the contents of the vessel over a period of three to five minutes while mixing continues. EDTA and NaCl are then added to the mixture while mixing continues for five more minutes at 75° C. The resulting mixture is cooled to 25 to 30° C. The final composition is packaged in appropriate containers.

### EXAMPLE 2

### Solution

A procedure similar to that of Example 1 is used to produce this solution.

| Ingredient | Amount (% by weight) |
|---|---|
| Compound having Formula IV | 0.35 |
| Mannitol | 4.5 |
| Benzakonium chloride (“BAK”) | 0.005 |
| Polysorbate 80 | 0.1 |
| EDTA | 0.05 |
| Sodium acetate | 0.03 |
| Acetic acid | 0.04 |
| Purified water | q.s. to 100 |

### EXAMPLE 3

### Solution

A procedure similar to that of Example 1 is used to produce this solution having the following composition.

| Ingredient | Amount (% by weight) |
|---|---|
| Compound having Formula IV | 0.2 |
| Dexamethasone | 0.1 |
| Hydroxypropylmethyl cellulose (“HPMC”) | 0.5 |
| Alexidine | 0.01 |
| Brij ® surfactant | 0.1 |
| EDTA | 0.1 |
| Citrate buffer (0.02M sodium citrate, pH = 5.0) | q.s. to 100 |

### EXAMPLE 4

### Solution

A procedure similar to that of Example 1 is used to produce this solution having the following composition.

| Ingredient | Amount (% by weight) |
|---|---|
| Compound 8 of Table 1 | 0.3 |
| Colecoxib | 0.15 |
| Propylene glycol | 0.5 |
| Alexidine | 0.01 |
| Tyloxapol | 0.1 |
| EDTA | 0.1 |
| Citrate buffer (0.02M sodium citrate, pH = 5) | q.s. to 100 |

### EXAMPLE 5

### Suspension

A procedure similar to that of Example 1 is used to produce this solution having the following composition.

| Ingredient | Amount (% by weight) |
|---|---|
| Compound having Formula IV | 0.3 |
| Triamcinolone, micronized USP | 0.2 |
| Hydroxyethyl cellulose | 0.25 |
| BAK | 0.01 |
| Tyloxapol | 0.05 |
| EDTA | 0.01 |
| NaCl | 0.3 |
| Na₂SO₄ | 1.2 |
| Sulfuric acid and/or NaOH | q.s. for pH adjustment to 5.5 |
| Citrate buffer (0.02M sodium citrate, | q.s. to 100 |
| pH = 5.0) | |

### EXAMPLE 6

### Emulsion

A modification of the procedure of Example 1 is used to produce this emulsion having the composition shown in the table below.

Polysorbate 60 (Tween® 60) is added to water in a first sterilized stainless steel jacketed vessel, equipped with a stirring mechanism, at a temperature of 50° C. to 60° C. in amounts corresponding the proportions shown in the table below. The resulting aqueous solution is heated to 61° C. to 75° C. At a temperature of 66° C., benzyl alcohol (a preservative) is added to the aqueous solution while mixing three to ten minutes. At a temperature of 75° C., appropriate amounts of the compound having Formula IV and loteprednole etabonate are added to Mygliol oil in a second sterilized vessel, also equipped with a stirring mechanism, over a period of three to five minutes while stirring continues. Sorbitan monostearate and cetyl stearyl alcohol are added to the oil mixture. The resulting oil mixture is heated to a temperature in the range from 62° C. to 75° C. The oil mixture is then added with vigorous mixing to the aqueous solution in the first vessel at a temperature of 66° C. over a period of three to five minutes. Sodium sulfate and sulfuric acid and/or sodium hydroxide are added to the mixture to adjust pH to 5.5. The resulting composition is cooled to 35° C. to 45° C. and homogenized by mixing with a high shear emulsifier or running through a homogenizer. The composition is further cooled to 25° C. to 30° C. The final composition is packaged in appropriate containers.

| Ingredient | Amount (% by weight) |
|---|---|
| Compound having Formula IV | 0.5 |
| Loteprednol etabonate | 0.2 |
| Polysorbate 60 | 1 |
| Sorbitan monostearate (an emulsifier) | 1.5 |
| Cetyl stearyl alcohol (an emulsion | 1.5 |
| stabilizer) | |
| Benzyl alcohol | 0.5 |
| Miglyol oil | 14.5 |
| Na₂SO₄ | 1.2 |
| Sulfuric acid and/or NaOH | q.s. for pH adjustment to 5.5 |
| Purified water | q.s. to 100 |

Typically, the oil used in an emulsion is a non-irritating emollient oil. Illustrative but non-limiting examples thereof include a mineral oil, vegetable oil, and a reformed vegetable oil of known composition. More specific but non-limiting examples of the oil can be selected from the group consisting of peanut oil, sesame seed oil, cottonseed oil, and a medium chain (C₆ to C₁₂) triglycerides (e.g., Miglyol Neutral Oils 810, 812, 818, 829, 840, etc., available from Huls America Inc.). Typical emulsifiers employed can be selected from the group consisting of sorbitan monostearate and polysorbate. Preferably, the emulsifiers are nonionic. The emulsifiers can be employed in an amount of 1.5 to 6.5% by weight of the composition, and preferably, 3 to 5% by weight of the composition. The hydrophobic phase of the emulsion can be in an amount of 15 to 25% by weight of the composition, and preferably, 18 to 22% by weight of the composition.

### EXAMPLE 7

### Emulsion

A procedure similar to that of Example 6 is used to produce this emulsion having the following composition.

| Ingredient | Amount (% by weight) |
|---|---|
| Compound 13 of Table 1 | 0.5 |
| Triamcinolone, micronized USP | 0.2 |
| Polysorbate 60 | 1 |
| Sorbitan monostearate | 1.5 |
| Cetyl stearyl alcohol | 1.5 |
| Benzyl alcohol | 0.5 |
| Miglyol oil | 14.5 |
| Na₂SO₄ | 1.2 |
| Sulfuric acid and/or NaOH | q.s. for pH adjustment to 5.5 |
| Purified water | q.s. to 100 |

### EXAMPLE 8

### Ointment

A procedure similar to that of Example 1 is used to produce this solution having the following composition.

| Ingredient | Amount (% by weight) |
|---|---|
| Compound having Formula IV | 0.3 |
| White petrolatum USP | 50 |
| Propylene glycol | 5 |
| Glycerin | 5 |
| Tween ® 20 | 2 |
| Vitamin E | 1 |
| BAK | 0.1 |
| Mineral oil | q.s. to 100 |

### EXAMPLE 9

### Ointment

A procedure similar to that of Example 1 is used to produce this solution having the following composition.

| Ingredient | Amount (% by weight) |
|---|---|
| Compound having Formula VI | 0.3 |
| Dexamethasone | 0.15 |
| White petrolatum USP | 50 |
| Propylene glycol | 5 |
| Glycerin | 5 |
| Tween ® 20 | 2 |
| Vitamin E | 1 |
| Vitamin D | 0.5 |
| BAK | 0.1 |
| Mineral oil | q.s. to 100 |

### EXAMPLE 10

### Tablet

The ingredients shown in the table below are blended together in a blender, such as a ribbon blender. Other types of blenders that are well known to people skilled in the art of powder mixing also can be used. The mixture is fed through a tableting press at conditions suitable for producing pharmaceutical tablets.

| Ingredient | Amount (% by weight) |
|---|---|
| Compound having Formula IV | 0.3 |
| Microcrystalline cellulose | 20 |
| Magnesium stearate | 2 |
| Mannitol | 65 |
| Starch | q.s. to 100 |

In one embodiment, the present invention provides a method for treating, reducing, or ameliorating an infection of an eye, ear or respiratory system, wherein such an infection is accompanied by an inflammation of the tissue in question. In one aspect, the method comprises administering one or more drops of a composition of the present invention to the eye, ear canal, nasal cavity, or back of the throat of a subject who has indication of infection or whose risk of infection is indicated. A composition of the present invention can also be formulated into a spray, which can be administered into the otic or nasal cavity of such a subject.

### Comparison of Side Effects of Glucocorticoids and Present Fluoroquinolones

One of the most frequent undesirable actions of a glucocorticoid therapy is steroid diabetes. The reason for this undesirable condition is the stimulation of gluconeogenesis in the liver by the induction of the transcription of hepatic enzymes involved in gluconeogenesis and metabolism of free amino acids that are produced from the degradation of proteins (catabolic action of glucocorticoids). A key enzyme of the catabolic metabolism in the liver is the tyrosine aminotransferase (“TAT”). The activity of this enzyme can be determined photometrically from cell cultures of treated rat hepatoma cells. Thus, the gluconeogenesis by a glucocorticoid can be compared to that of a fluoroquinolone disclosed herein by measuring the activity of this enzyme. For example, in one procedure, the cells are treated for 24 hours with the test substance (a fluoroquinolone or glucocorticoid), and then the TAT activity is measured. The TAT activities for the selected fluoroquinolone and glucocorticoid are then compared. Other hepatic enzymes can be used in place of TAT, such as phosphoenolpyruvate carboxykinase, glucose-6-phosphatase, or fructose-2,6-biphosphatase. Alternatively, the levels of blood glucose in an animal model may be measured directly and compared for individual subjects that are treated with a glucocorticoid for a selected condition and those that are treated with a fluoroquinolone for the same condition.

Another undesirable result of glucocorticoid therapy is GC-induced cataract. The cataractogenic potential of a compound or composition may be determined by quantifying the effect of the compound or composition on the flux of potassium ions through the membrane of lens cells (such as mammalian lens epithelial cells) in vitro. Such an ion flux may be determined by, for example, electrophysiological techniques or ion-flux imaging techniques (such as with the use of fluorescent dyes). An exemplary in-vitro method for determining the cataractogenic potential of a compound or composition is disclosed in U.S. Patent Application Publication 2004/0219512, which is incorporated herein by reference.

Still another undesirable result of glucocorticoid therapy is hypertension. Blood pressure of similarly matched subjects treated with glucocorticoid and a fluoroquinolone of the present invention for an inflammatory condition may be measured directly and compared.

Yet another undesirable result of glucocorticoid therapy is increased intraocular pressure (“IOP”) in the subject. IOP of similarly matched subjects treated with glucocorticoid and a fluoroquinolone of the present invention for a condition may be measured directly and compared.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A method for modulating an inflammation in a subject, the method comprising administering to the subject a composition comprising an effective amount of a fluoroquinolone having Formula I, II, III, IV, V, VI, VII, or VIII, or a salt thereof 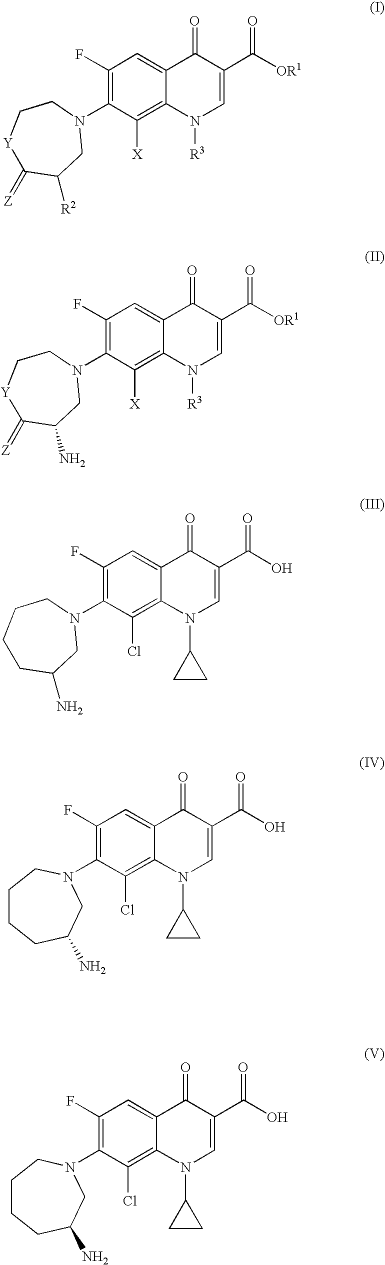 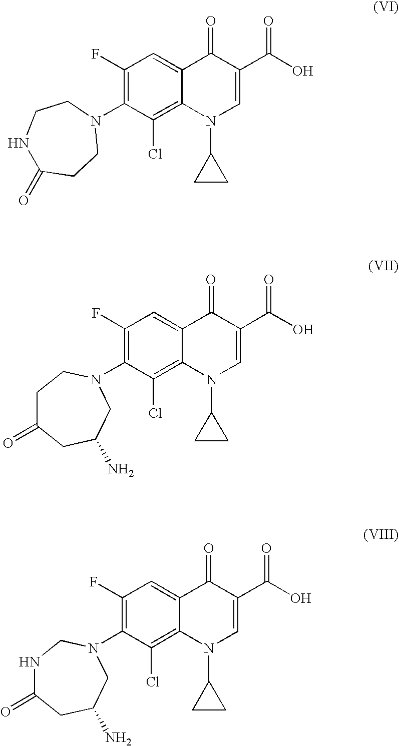
wherein R¹ is selected from the group consisting of hydrogen, unsubstituted lower alkyl groups, substituted lower alkyl groups, cycloalkyl groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, and groups that can be hydrolyzed in living bodies;
R² is selected from the group consisting of hydrogen, unsubstituted amino group, and amino groups substituted with one or two lower alkyl groups;
R³ is selected from the group consisting of hydrogen, unsubstituted lower alkyl groups, substituted lower alkyl groups, cycloalkyl groups, unsubstituted lower alkoxy groups, substituted lower alkoxy groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, unsubstituted C₅-C₂₄ aryloxy groups, substituted C₅-C₂₄ aryloxy groups, unsubstituted C₅-C₂₄ heteroaryloxy groups, substituted C₅-C₂₄ heteroaryloxy groups, and groups that can be hydrolyzed in living bodies;
X is selected from the group consisting of halogen atoms;
Y is selected from the group consisting of CH₂, O, S, SO, SO₂, and NR⁴, wherein R⁴ is selected from the group consisting of hydrogen, unsubstituted lower alkyl groups, substituted lower alkyl groups, and cycloalkyl groups; and
Z is selected from the group consisting of oxygen and two hydrogen atoms.

2. The method of claim 1, wherein said inflammation is selected from the group consisting of uveitis, vernal keratoconjunctivitis, or inflammation associated with contact lens-associated corneal infiltrates.

3. The method of claim 1, wherein said inflammation comprises a sequela of an infection.

4. The method of claim 3, wherein said infection comprises an ocular or ophthalmic infection.

5. The method of claim 1, wherein R¹ is selected from the group consisting of hydrogen, C₁-C₅ substituted and unsubstituted alkyl groups, C₃-C₁₀ cycloalkyl groups, C₅-C₁₄ substituted and unsubstituted aryl groups, C₅-C₁₄ substituted and unsubstituted heteroaryl groups, and groups that can be hydrolyzed in living bodies. In one embodiment, R¹ is selected from the group consisting of C₁-C₅ substituted and unsubstituted alkyl groups.

6. The method of claim 1, wherein R² is selected from the group consisting of unsubstituted amino group and amino groups substituted with one or two C₁-C₅ alkyl groups.

7. The method of claim 1, wherein R³ is selected from the group consisting of hydrogen, C₁-C₅ substituted and unsubstituted alkyl groups, C₃-C₁₀ cycloalkyl groups, C₁-C₅ substituted and unsubstituted alkoxy groups, C₅-C₁₄ substituted and unsubstituted aryl groups, C₅-C₁₄ substituted and unsubstituted heteroaryl groups, and C₅-C₁₄ substituted and unsubstituted aryloxy groups.

8. The method of claim 1, wherein R³ is selected from the group consisting of C₃-C₁₀ cycloalkyl groups.

9. The method of claim 1, wherein X is selected from the group consisting of Cl, F, and Br.

10. The method of claim 1, wherein X is Cl.

11. The method of claim 5, wherein X is F.

12. The method of claim 10, wherein Y is CH₂.

13. The method of claim 10, wherein Z comprises two hydrogen atoms.

14. The method of claim 1, wherein Y is NH, Z is O, and X is Cl.

15. The method of claim 1, wherein the composition is administered into the subject topically, orally, subcutaneously, or systemically.

16. The method of claim 1, wherein the composition comprises a solution, emulsion, dispersion, suspension, ointment, or gel.

17. The method of claim 16, wherein the fluoroquinolone or salt thereof is present in an amount from about 0.0001% to 10% by weight of the composition.

18. The method of claim 17, wherein the composition further comprises a carrier and a material selected from the group consisting of preservatives, surfactants, adjuvants, antioxidants, tonicity adjusters, viscosity modifiers, solubility enhancers, and combinations thereof.

19. The method of claim 17, wherein the composition further comprises a non-steroidal anti-inflammatory drug.

20. A method for modulating an inflammation in a subject, the method comprising administering to the subject a composition comprising an effective amount of a fluoroquinolone having Formula IV or a salt thereof

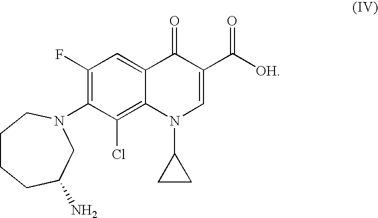

21. The method of claim 20, wherein the inflammation comprises an ocular or ophthalmic inflammation.

22. The method of claim 21, wherein said administering comprises a topical or intraocular administration.

23. The method of claim 21, wherein said inflammation comprises a sequela of an infection.

24. A method for treating, controlling, reducing, or ameliorating an ocular or ophthalmic infection and an inflammatory sequela thereof in a subject, the method comprising administering to the subject a composition comprising an effective amount of a fluoroquinolone having Formula IV or a salt thereof

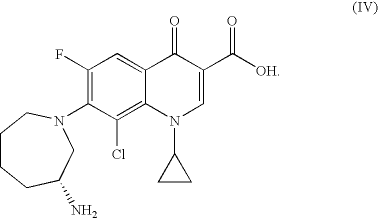

25. A method for modulating an inflammation in a subject, the method comprising administering to the subject a composition comprising an effective amount of a fluoroquinolone having Formula VI or a salt thereof

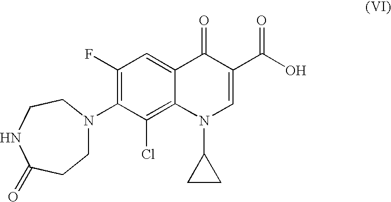

26. A method for treating, controlling, reducing, or ameliorating an ocular or ophthalmic infection and an inflammatory sequela thereof in a subject, the method comprising administering to the subject a composition comprising an effective amount of a fluoroquinolone having Formula VI or a salt thereof

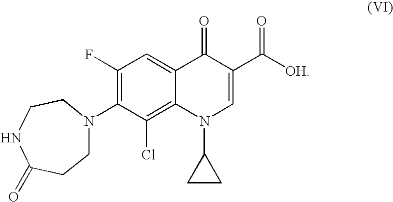

27. Use of a fluoroquinolone having Formula I, II, III, IV, V, VI, VII, or VIII for a preparation of a medicament for modulating an inflammation in a subject.

28. A pharmaceutical composition comprising a fluoroquinolone having Formula I, II, III, IV, V, VI, VII, or VIII, wherein said fluoroquinolone is present in an amount effective to modulate an inflammation.

29. The pharmaceutical composition of claim 28, wherein said inflammation comprises a sequela of an infection.

30. The pharmaceutical composition of claim 29, wherein said infection comprises an ocular or ophthalmic infection.
